(19) United States
(12) Patent Application Publication
(10) Pub. No.: US 2022/0068484 A1
(43) Pub. Date: Mar. 3, 2022

(54) SYSTEMS AND METHODS FOR USING TRAINED PREDICTIVE MODELING TO REDUCE MISDIAGNOSES OF CRITICAL ILLNESSES

(71) Applicant: Evernorth Strategic Development, Inc., St. Louis, MO (US)

(72) Inventors: Daniel J. Greden, West Hartford, CT (US); David Spangler, Hilton Head, SC (US); Bhuvana Sagar, Spring, TX (US); Richard S. Emery, Portland, OR (US); Stephen A. Hamilton, Edmond, OK (US); Eric J. Gratias, Savannah, GA (US)

(21) Appl. No.: 17/462,169

(22) Filed: Aug. 31, 2021

Related U.S. Application Data
(60) Provisional application No. 63/072,605, filed on Aug. 31, 2020.

(51) Int. Cl. G16H 50/20; G06Q 40/08; G16H 10/60; G16H 70/60; G16H 40/20; G16H 40/67; G16H 50/70

(52) U.S. Cl. CPC G16H 50/20; G06Q 40/08; G16H 10/60; G16H 50/70; G16H 40/20; G16H 40/67; G16H 70/60

(57) ABSTRACT

A trained predictive server is provided for determining that a diagnosis and treatment plan is inaccurate. The trained predictive server includes a processor configured to receive a set of prior authorization (PA) data associated with a medical claim for a patient, and determine that the set of PA data indicates that the medical claim is associated with a qualifying critical illness. The processor is further configured to extract component data from the set of PA data, and apply the extracted component data to a trained predictive model associated with the qualifying critical illness to determine whether the medical claim is associated with an inaccurate diagnosis and treatment plan. Upon determining that the medical claim is associated with an inaccurate diagnosis and treatment plan, the processor is configured to generate a request for a consulting review of the diagnosis and treatment plan using the set of PA data.

### CROSS-REFERENCE TO RELATED APPLICATION

This application claims the benefit of U.S. Provisional Patent Application No. 63/072,605, filed Aug. 31, 2020, which is incorporated by reference herein in its entirety.

### FIELD

The field relates to systems and methods for using trained predictive models to predict whether diagnoses of critical illnesses are inaccurate and require a second opinion. The field also relates to the use of prior authorization (PA) data and related systems to facilitate the training of such predictive models.

### BACKGROUND

Many known critical illnesses, including chronic illnesses and terminal illnesses, are susceptible to misdiagnosis. There are many reasons for such misdiagnoses. Such critical illnesses are being actively researched and studied, making accurate assessment of the illnesses difficult to determine for healthcare providers who are not actively following the state-of-the-art related to each illness. Further, many such critical illnesses are rare and/or complex and make it difficult for even some specialist healthcare providers to readily understand. Additionally, reliable diagnosis of such critical illnesses may depend upon the availability of testing and diagnostic tools that may not be available to all healthcare providers.

At least some misdiagnoses may lead to adverse outcomes, including death. For example, failure to accurately diagnose an illness could delay or impede the creation, development, and/or implementation of an appropriate treatment plan. Left untreated, at least some illnesses tend to get worse over time. Moreover, at least some misdiagnoses could result in a treatment plan that is ineffective or even counterproductive. Further, because treatment can be expensive and resource intensive, such misdiagnoses may contribute to unnecessary waste of medical resources and financial resources of patients and insurers.

### BRIEF SUMMARY

Examples described herein enable predicting whether diagnoses or treatments of critical illnesses are inaccurate. In one aspect, a trained predictive server is provided for determining that a diagnosis and treatment plan is inaccurate. The trained predictive server includes a processor and a memory. The processor is configured to receive a set of prior authorization (PA) data associated with a medical claim for a patient, and determine that the set of PA data indicates that the medical claim is associated with a qualifying critical illness. The processor is further configured to extract component data from the set of PA data, and apply the extracted component data to a trained predictive model associated with the qualifying critical illness to determine whether the medical claim is associated with an inaccurate diagnosis and treatment plan. Upon determining that the medical claim is associated with an inaccurate diagnosis and treatment plan, the processor is configured to generate a request for a consulting review of the diagnosis and treatment plan using the set of PA data.

In another aspect, a method is provided for determining that a diagnosis and treatment plan is inaccurate. The method is performed by a trained predictive server including a processor and a memory. The method includes receiving a set of prior authorization (PA) data associated with a medical claim for a patient, and determining that the set of PA data indicates that the medical claim is associated with a qualifying critical illness. The method further includes extracting component data from the set of PA data, and applying the extracted component data to a trained predictive model associated with the qualifying critical illness to determine whether the medical claim is associated with an inaccurate diagnosis and treatment plan. Upon determining that the medical claim is associated with an inaccurate diagnosis and treatment plan, a request for a consulting review of the diagnosis and treatment plan is generated using the set of PA data.

In yet another aspect, a trained predictive system is provided for determining that a diagnosis and treatment plan is inaccurate. The trained predictive system includes a first claim database server including a database processor and a database memory. The database memory includes a set of prior authorization (PA) data associated with a medical claim for a patient. The database processor is configured to determine that the set of PA data indicates that the medical claim is associated with a qualifying critical illness. The trained predictive system further includes a trained predictive server in communication with the first claim database server. The trained predictive server includes a processor and a memory. The processor is configured to receive the set of PA data associated with the medical claim for the patient from the first claim database server. The processor is further configured to extract component data from the set of PA data, and apply the extracted component data to a trained predictive model associated with the qualifying critical illness to determine whether the medical claim is associated with an inaccurate diagnosis and treatment plan. The database processor is further configured to receive an indication that the medical claim is associated with the inaccurate diagnosis and treatment plan from the trained predictive server, and generate a request for a consulting review of the diagnosis and treatment plan using the set of PA data.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 is a functional block diagram of an example insurance claim processing system;

FIG. 2 is a functional block diagram of an example computing device;

FIG. 3 is a functional block diagram of an example trained predictive system that may be deployed within the system of FIG. 1 using the computing device shown in FIG. 2; and

FIG. 4 is a flow diagram representing an example method for determining that a diagnosis and treatment plan is inaccurate from the perspective of the trained predictive server shown in FIG. 3.

In the drawings, reference numbers may be reused to identify similar and/or identical elements.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

As used herein, the term “feature selection” refers to the process of selecting a subset of relevant features (e.g., variables or predictors) that are used in the machine learning system to define data models. Feature selection may alternatively be described as variable selection, attribute selection, or variable subset selection. The feature selection process of the machine learning system described herein allows the machine learning system to simplify models to make them easier to interpret, reduce the time to train the systems, reduce overfitting, enhance generalization, and avoid problems in dynamic optimization. The data models described herein may include known data models and/or novel data models.

The machine learning systems and methods described herein are configured to address known technological problems confronting computing systems and networks that process data sets, specifically the lack of known static relationships between data sets and certain data characteristics.

In many examples, the reliability and accuracy of diagnostic and treatment determinations are important aspects of claim processing. In healthcare systems, healthcare providers typically provide (directly or indirectly) data associated with a patient upon making a diagnosis and determining a treatment plan. Typically, a set of relevant data (e.g., diagnosis, treatment plan) is provided in the context of a prior authorization (PA) request. PA requests are typically required by healthcare insurers after a physician prescribes such a treatment plan in order to confirm that the proposed treatment is covered by the insurer. Healthcare providers generate data related to treatment outcomes. Healthcare insurers also have access, directly or indirectly, to data related to treatment outcomes.

The consequences of an inaccurate diagnosis and/or treatment plan may dramatically affect the health and outcome of a patient, as well as incur significant financial and resource costs. Such consequences are elevated in the context of critical illnesses, and particularly in the context of chronic and terminal illnesses such as cancer. Moreover, while certain data relationships associated with the risk of misdiagnosis and/or mistreatment are known, assessing the reliability and accuracy of diagnoses and treatments associated with at least some illnesses is indeterminate or exacerbated because methodology for diagnoses and treatments of such illnesses is constantly evolving. As such, while static models may be applied using the methods and systems described herein, machine learning models are also contemplated and described herein. In this manner, the examples described herein are configured to systematically improve over time as illnesses are better understood and innovations, discoveries, and other developments in diagnoses and treatments continue to emerge.

The described machine learning systems and methods solve a technological problem related to unreliable data that cannot be otherwise resolved using known methods and technologies. In particular, the proposed approach of using machine learning to train a model to assess the diagnostic or treatment determinations is a significant technological improvement in the technological field of health and data sciences. By determining that a medical claim is associated with an inaccurate diagnosis or treatment plan as described herein, at least some medical claims can be systematically managed, enabling computing systems to reduce or mitigate the amount of storage space, bandwidth, processing power, etc. used on unreliable data (e.g., inaccurate diagnosis or treatment plan) and ultimately improve workflows, runtime performance, and data quality. Further, the proposed approach includes active re-training to ensure predictive accuracy and provide real-world benefits. For example, reducing the number of false negatives for illnesses may lead to earlier detection, reliable diagnoses, earlier treatment, appropriate treatment plans, and improved outcomes, and reducing the number of false positives may reduce the need for confirmatory testing and mitigate the risk of improper and/or unwarranted treatment. Moreover, increasing predictivity enables patients, healthcare providers, and insurers to rely on a diagnosis and treatment plan with greater confidence.

Generally, the systems and methods described apply a cyclical process of (a) defining a cohort for pattern identification that may be defined based on diagnosed illness types aggregating across at least one of: (i) patients, (ii) providers, and (iii) geographical regions; (b) identifying prior authorization (PA) or claim data associated with relevant critical claims within the cohort; (c) collecting or extracting data from PA data for the cohort; (d) determining patterns or relationships between PA data and outcomes for the cohort; (e) creating a data model based on the patterns or relationships; and (f) refining the data model with new data. The data model may be applied by the predictive server to PA data for incoming claims to identify likely misdiagnoses or mistreatment, and to recommend a clinical consultation (or second opinion) to reassess the diagnoses or treatment plan for such identified claims.

As described above, the systems and methods define cohorts based on diagnosed illness (or condition) type aggregating across a variety of groupings. In an example embodiment, the relevant illnesses or conditions that define the cohorts include at least one of breast cancer, non-small cell lung cancer, bone cancer, soft tissue sarcoma, colorectal cancer, central nervous system cancer, Hodgkin disease, and multiple myeloma. In the example embodiment, these conditions are selected because they are complex illnesses with a variety of possible underlying diagnoses and treatments. In other examples, the cohorts may include other chronic or terminal illnesses. It is contemplated that the systems and methods described may be relevant for a variety of such illnesses including autoimmune diseases and neurological diseases.

As described above, the machine learning models analyze the underlying PA and outcome data to determine certain data models that can be used to identify possible misdiagnoses or mistreatment based on composite PA data. Such models may include some of the following: (i) clinical indications with a high rate of misdiagnosis; (ii) clinical indications with a high rate of redirection (i.e., the diagnoses were not complete); (iii) healthcare providers associated with more frequent determinations of incorrect diagnoses or treatments; (iv) institutions associated with more frequent determinations of incorrect diagnoses or treatments; (vi) patient information including demographic data (e.g., age, gender, sex), relevant health biometric data, geographic data, and history data associated with frequent determinations of incorrect diagnoses or treatments. In many examples, the systems and methods described may create additional data models with combinations of such models. In some examples, the systems and methods may incorporate additional extrinsic data to create models including, for example, clinical research data, imaging data, and other health system data.

A trained predictive system for determining that a diagnosis and treatment plan is inaccurate is provided. The trained predictive system includes a first claim database server with a database processor and a database memory. The first claim database server includes a set of prior authorization (PA) data associated with a medical claim for a patient. In operation, the first claim database server receives requests from computing devices associated with healthcare providers including, for example, provider computing devices, hospital computing devices, and clinic computing devices. More specifically, when a provider makes a determination of a diagnosis and/or treatment plan for a particular patient, a PA request is typically submitted to an insurer associated with the first claim database. The PA request may include a variety of information related to the diagnosis and treatment of a particular patient. For example, PA requests may include some or all of: diagnostic data, treatment data, patient demographic data, patient geographic data, patient socioeconomic data, healthcare provider data, and healthcare provider reputation data. PA requests may also include relevant information for claim processing including insurer data, insurer identifiers, insured data, insured identifiers, and coverage data.

The trained predictive system also includes a trained predictive server that is in communication with the first claim database server. The trained predictive server includes a processor and a memory. The trained predictive server is configured to receive the set of prior authorization (PA) data associated with the medical claim for the patient from the first claim database. In some examples, the first claim database server and/or trained predictive server may be configured to determine whether the set of PA data indicates that the medical claim is associated with a qualifying critical illness. For example, the first claim database server and/or trained predictive server may identify or receive a list of qualifying critical illnesses from a storage device (e.g., a first data warehouse server) and apply the list to the set of PA data to determine whether the set of PA data indicates that the medical claim is associated with a qualifying critical illness, such as breast cancer, non-small cell lung cancer, bone cancer, soft tissue sarcoma, colorectal cancer, central nervous system cancer, Hodgkin disease, and multiple myeloma. The list of qualifying critical illnesses may include any illness or condition that allows or enables the trained predictive system to operate as described herein.

The trained predictive server is configured to extract component data from the set of PA data and apply the extracted component data to a trained predictive model to determine whether the medical claim is associated with an inaccurate diagnosis and treatment plan. In at least some examples, the trained predictive server identifies or receives a list of features for extraction, and extracts the component data, based on the list of features, from the set of PA data. The list of features may be predefined or derived. In some examples, the list of features are determined to include those features that are likely to indicate or influence an inaccurate diagnosis or treatment plan. The list of features may include, without limitation, diagnostic data, treatment data, patient demographic data, patient geographic data, patient socioeconomic data, healthcare provider data, and/or healthcare provider reputation data.

In some examples, the trained predictive server may quantify the extracted component data to determine a probability score or computation that describes or indicates a likelihood of whether the medical claim is associated with an inaccurate diagnosis and treatment plan. Probability scoring or computation may be valuable when the determination of inaccuracy is not obtainable in a binary fashion. The probability score may be compared against one or more predetermined thresholds to evaluate inaccuracies in the diagnosis and treatment plan and then determine an appropriate follow-up action. For example, a predetermined threshold may be set at 80% (or other suitable percentage) to distinguish medical claims that require a second opinion (e.g., because they are at least 80% likely to be associated with an inaccurate diagnosis and treatment plan) from medical claims that do not require a second opinion (e.g., because they are less than 80% likely to be associated with an inaccurate diagnosis and treatment plan). Additionally or alternatively, a predetermined threshold may be set at 21% (or other suitable percentage) to distinguish medical claims that are approved for processing (e.g., because they are less than 21% likely to be associated with an inaccurate diagnosis and treatment plan) from medical claims that are not (yet) approved for processing (e.g., because they are at least 21% likely to be associated with an inaccurate diagnosis and treatment plan). In some examples, the trained predictive server may be configured to generate or transmit an alert to request additional input (e.g., from a systems analyst, physician, or other technical or health care professional) regarding whether a second opinion is required and/or a medical claim is approved for processing when the medical claim is at least 21% (or other suitable percentage) and less than 80% (or other suitable percentage) likely to be associated with an inaccurate diagnosis and treatment plan. In operation, any suitable threshold (and any number of thresholds) may be applied depending on, e.g., the medical claim, the diagnosis and treatment plan, or other criteria being evaluated, and the comparisons between one or more thresholds and likelihoods may include statistical analyses (e.g., standard deviations), and may utilize rounding or other suitable approximations.

Upon determining that the medical claim is associated with an inaccurate diagnosis and treatment plan, the trained predictive server may generate a request for a consulting review of the diagnosis and treatment plan using the set of PA data. Additionally or alternatively, the request may be generated by the first claim database server. In any case, a consulting review provider may be identified based on the set of PA data, and the request may be transmitted to a computing device associated with the consulting review provider. In some examples, the first claim database server and/or trained predictive server may be configured to identify a list of candidate consulting review providers, wherein each candidate consulting review provider is associated with candidate provider reputation data and candidate provider location data, and to identify the consulting review provider from the list of candidate consulting review providers based on a comparison of the set of PA data to the candidate provider reputation data and the candidate provider location data.

A machine learning system is provided for training a predictive model to determine whether a diagnosis and treatment plan for a patient is inaccurate. For example, the machine learning system may apply a method to create and/or train the data models described herein. In some examples, the machine learning system includes the trained predictive server and a first data warehouse server in communication with the trained predictive server. The first data warehouse server may include a data warehouse processor and a data warehouse memory including data that may be used to create and/or train one or more data models. The data warehouse memory may include, for example, a plurality of historical prior authorization (PA) data and a plurality of result data associated with the historical PA data. Each historical PA data and associated result data is further associated with a patient claim. The result data may indicate whether each associated patient claim successfully processed.

A set of feature data may be extracted from each of the historical PA data. In at least some examples, the trained predictive server determines a list of features for extraction and extracts the feature data, based on the list of features, from the plurality of historical PA data. In some examples, the list of features are determined to include those features that are likely to indicate or influence an inaccurate diagnosis or treatment plan. Historical PA data that describes or indicates an inaccurate diagnosis or treatment plan may be associated, for example, with result data that describes or indicates an adverse outcome. The list of features may include, without limitation, diagnostic data, treatment data, patient demographic data, patient geographic data, patient socioeconomic data, healthcare provider data, and/or healthcare provider reputation data.

In some examples, the trained predictive server may be configured to apply the extracted feature data to a data model to determine whether the data model is reliable (e.g., whether the data model is configured to accurately determine whether a diagnosis and treatment plan associated with a medical claim is accurate or inaccurate). The trained predictive server may implement a feature selection process using one or more machine learning algorithms to facilitate improving a reliability of the data model. In addition to selecting one or more features, the machine learning algorithms may also determine one or more algorithms for use in selecting the features.

The trained predictive server may also be configured to extract a set of retraining feature data from each of the historical PA data and retrain one or more data models based on the retraining feature data. For example, the trained predictive server may identify or receive a first portion of the plurality of historical PA data and an associated first portion of the plurality of result data for use in creating and/or training one or more data models based on the first portion of the plurality of historical PA data and the first portion of the plurality of result data. The trained predictive server may then identify or receive a second portion of the plurality of historical PA data and an associated second portion of the plurality of result data for use in “retraining” one or more data models based on the second portion of the plurality of historical PA data and the second portion of the plurality of result data. In this manner, models can be iteratively or dynamically retrained as new historical PA data and associated result data is generated.

Generally, the systems and methods described herein are configured to perform at least the following steps: receiving a set of prior authorization (PA) data associated with a medical claim for a patient; identifying a list of qualifying critical illnesses from a storage device in communication with the trained predictive server; applying the list to the set of PA data to determine whether the set of PA data indicates that the medical claim is associated with a qualifying critical illness; determining that the set of PA data indicates that the medical claim is associated with a qualifying critical illness; identifying a list of features for extraction; extracting component data from the set of PA data; applying the extracted component data to the trained predictive model to determine whether the medical claim is associated with an inaccurate diagnosis and treatment plan; determining whether the medical claim is associated with an inaccurate diagnosis and treatment plan; determining a likelihood of whether the medical claim is associated with an inaccurate diagnosis and treatment plan; comparing the likelihood of whether the medical claim is associated with an inaccurate diagnosis and treatment plan against a predetermined threshold to evaluate inaccuracies in the diagnosis and treatment plan; identifying a list of candidate consulting review providers, wherein each candidate consulting review partner is associated with candidate provider reputation data and candidate provider location data; identifying a consulting review provider based on the set of PA data; identifying the consulting review provider from the list of candidate consulting review providers based on a comparison of the set of PA data to the candidate provider reputation data and the candidate provider location data; generating a request for a consulting review of the diagnosis and treatment plan using the set of PA data (e.g., on condition that the likelihood of whether the medical claim is associated with an inaccurate diagnosis and treatment plan is greater than an upper predetermined threshold); transmitting the request to a computing device associated with the consulting review provider; generating a request that the medical claim associated with the set of PA data be processed (e.g., on condition that the likelihood of whether the medical claim is associated with an inaccurate diagnosis and treatment plan is less than a lower predetermined threshold); generating or transmitting an alert to a secondary device to request input (e.g., on condition that the likelihood of whether the medical claim is associated with an inaccurate diagnosis and treatment plan is greater than a lower predetermined threshold and less than an upper predetermined threshold); determining a list of features for extraction; extracting feature data, based on the list of features, from a plurality of historical PA data; applying the extracted feature data to the data model to determine whether the data model is reliable; generating or modifying a data model based on the plurality of historical PA data and a plurality of result data associated with the plurality of historical PA data; receiving a first portion of the plurality of historical PA data and an associated first portion of the plurality of result data; and/or receiving a second portion of the plurality of historical PA data and an associated second portion of the plurality of result data.

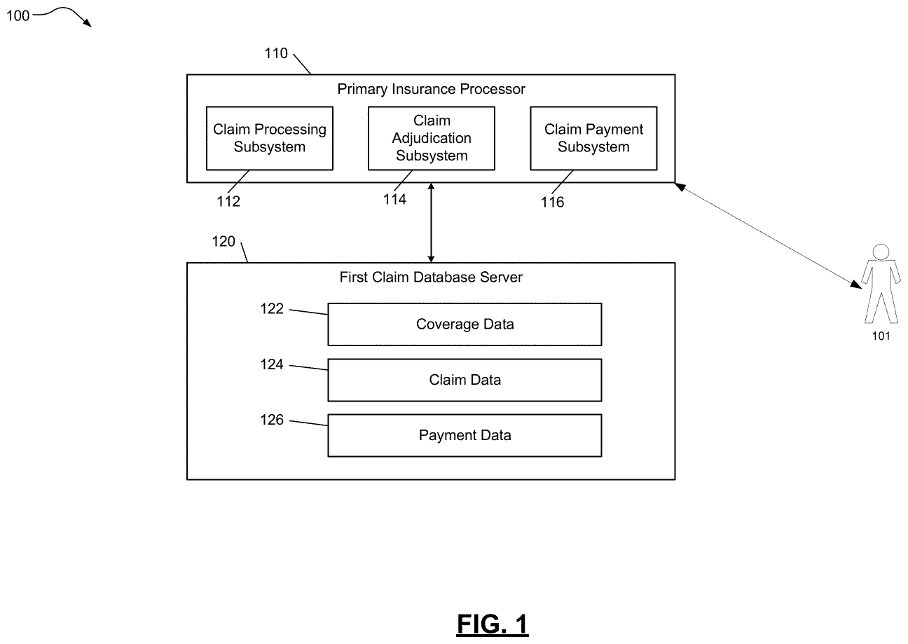

FIG. 1 is a functional block diagram of an example insurance claim processing system 100. Insurance processor system 110 includes subsystems 112, 114, and 116 capable of providing claim processing, claim adjudication, and claim payment respectively. First claim database server 120 includes necessary information on its underlying database. Specifically, first claim database server 120 includes coverage data 122, claim data 124, and payment data 126.

In operation, users such as user 101 may interact with insurance processor system 110. User 101 may be a healthcare provider, a patient, or any other suitable user involved in creating or reviewing claims. As described herein, in at least some examples, user 101 is a healthcare provider rendering diagnoses and/or treatment plans that are submitted in PA data to an insurer associated with insurance processor system 110.

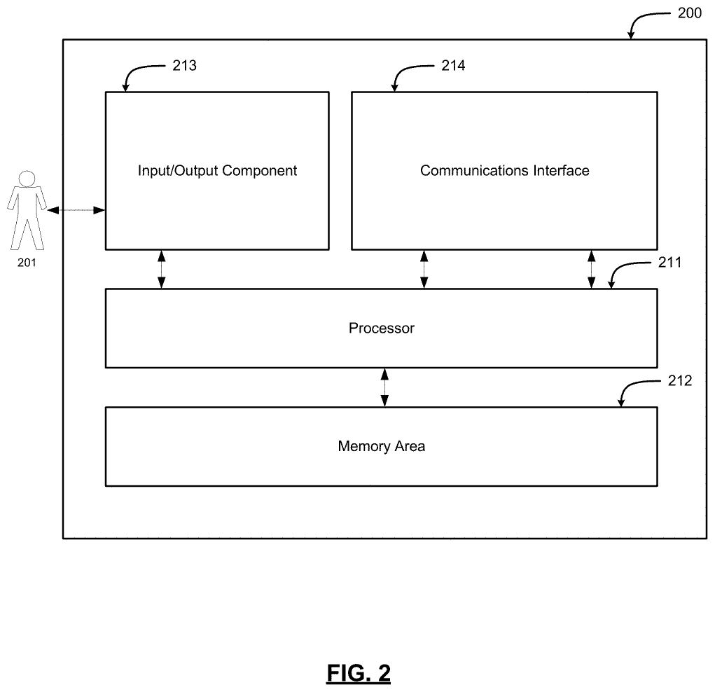

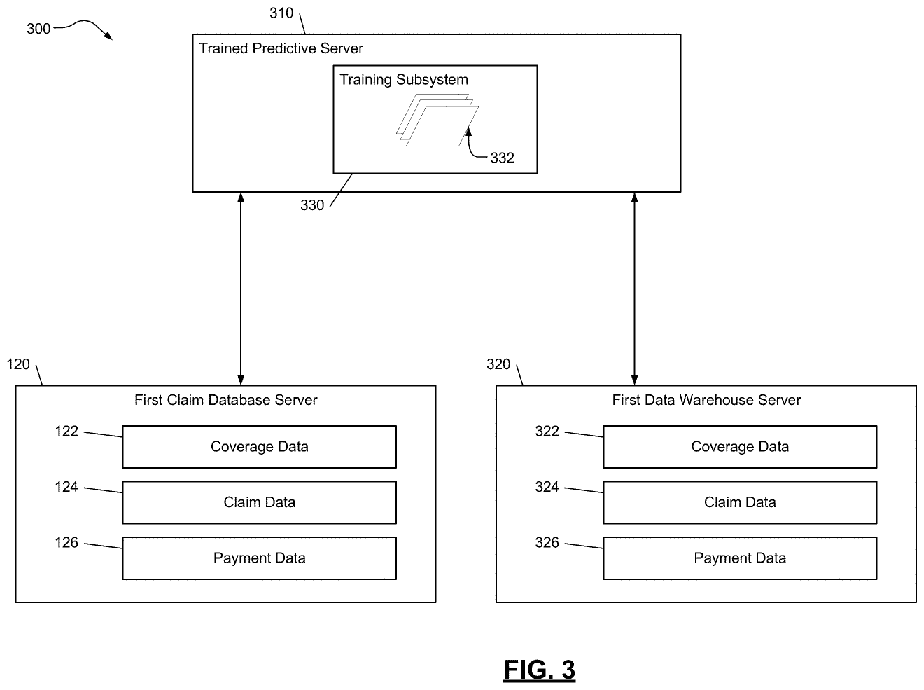

FIG. 2 is a functional block diagram of an example computing device that may be used in the trained predictive system described herein. Specifically, computing device 200 illustrates an example configuration of a computing device for the systems shown herein, and particularly in FIGS. 1 and 3. Computing device 200 illustrates an example configuration of a computing device operated by a user 201 in accordance with one embodiment of the present invention. Computing device 200 may include, but is not limited to, first claim database server, trained predictive server, first data warehouse server, and first predictive server, and other user systems, and other server systems. Computing device 200 may also include servers, desktops, laptops, mobile computing devices, stationary computing devices, computing peripheral devices, smart phones, wearable computing devices, medical computing devices, and vehicular computing devices. In some variations, computing device 200 may be any computing device capable of the described methods for predicting that PA data includes an incorrect diagnosis or treatment plan. In some variations, the characteristics of the described components may be more or less advanced, primitive, or non-functional.

In an example embodiment, computing device 200 includes a processor 211 for executing instructions. In some embodiments, executable instructions are stored in a memory area 212. Processor 211 may include one or more processing units, for example, a multi-core configuration. Memory area 212 is any device allowing information such as executable instructions and/or written works to be stored and retrieved. Memory area 212 may include one or more computer readable media.

Computing device 200 also includes at least one input/output component 213 for receiving information from and providing information to user 201 (e.g., user 101). In some examples, input/output component 213 may be of limited functionality or non-functional as in the case of some wearable computing devices. In other examples, input/output component 213 is any component capable of conveying information to or receiving information from user 201. In some embodiments, input/output component 213 includes an output adapter such as a video adapter and/or an audio adapter. Input/output component 213 may alternatively include an output device such as a display device, a liquid crystal display (LCD), organic light emitting diode (OLED) display, or “electronic ink” display, or an audio output device, a speaker or headphones. Input/output component 213 may also include any devices, modules, or structures for receiving input from user 201. Input/output component 213 may therefore include, for example, a keyboard, a pointing device, a mouse, a stylus, a touch sensitive panel, a touch pad, a touch screen, a gyroscope, an accelerometer, a position detector, or an audio input device. A single component such as a touch screen may function as both an output and input device of input/output component 213. Input/output component 213 may further include multiple sub-components for carrying out input and output functions.

Computing device 200 may also include a communications interface 214, which may be communicatively coupleable to a remote device such as a remote computing device, a remote server, or any other suitable system. Communication interface 214 may include, for example, a wired or wireless network adapter or a wireless data transceiver for use with a mobile phone network, Global System for Mobile communications (GSM), 3G, 4G, or other mobile data network or Worldwide Interoperability for Microwave Access (WIMAX). Communications interface 214 is configured to allow computing device 200 to interface with any other computing device or network using an appropriate wireless or wired communications protocol such as, without limitation, BLUETOOTH®, Ethernet, or IEEE 802.11. Communications interface 214 allows computing device 200 to communicate with any other computing devices with which it is in communication or connection.

FIG. 3 is a functional block diagram of a trained predictive system 300 that may be deployed within system 100 (shown in FIG. 1) using the computing device 200 (shown in FIG. 2). Specifically, trained predictive system 300 includes a trained predictive server 310 which is in communication with at least first claim database server 120, which may be associated with a first healthcare provider. For example, once the first healthcare provider makes a determination of a diagnosis and/or treatment plan for a particular patient, the first claim database server 120 may include PA data associated with the patient (e.g., diagnostic data, treatment data, patient demographic data, patient geographic data, patient socioeconomic data) and/or the first healthcare provider (e.g., healthcare provider data, healthcare provider reputation data). The trained predictive server 310 is also in communication with a first data warehouse server 320, which includes a plurality of historical prior authorization (PA) data associated with a plurality of patients and/or healthcare providers as well as a plurality of result data associated with the historical PA data. For example, as shown in FIG. 3, the first data warehouse server 320 may include coverage data 322, claim data 324, and payment data 326. In this manner, coverage data 122 and 322 and claim data 124 and 324 may include the relevant PA data and outcome data described herein.

Trained predictive server 310 includes subsystems capable of performing the methods described herein and, more specifically, training subsystem 330 generates and/or defines one or more models 332 for predicting misdiagnoses and mistreatment. Models 332 may be generated using data in first data warehouse server 320 (e.g., coverage data 322, claim data 324). Trained predictive server 310 is configured to apply such models 332 to data in first claim database server 120 and, more specifically, to PA data included therein (e.g., coverage data 122, claim data 124) for determining whether a diagnosis and/or treatment plan is accurate.

Models 332 may be generated and/or modified to improve the reliability and accuracy of diagnostic and treatment determinations. For example, the trained predictive server 310 may create and/or refine one or more models 332 by applying a cyclical process of defining a cohort; collecting or extracting historical PA data for the cohort, as well as the corresponding result data; determining patterns or relationships between the historical PA data and the corresponding result data, and selecting one or more features (e.g., variables or predictors) that have a correlation with inaccurate diagnoses and treatment plans. Such features are selected based on their propensity to indicate or influence an inaccurate diagnosis or treatment plan. For example, the trained predictive server 310 may conduct a correlation analysis between the historical PA data and result data and select one or more features based on a correlation coefficient between such features and result data describing or indicating an adverse outcome. Additionally or alternatively, one or more features may be selected based on the availability of testing and diagnostic tools. One or more machine learning algorithms may be used to select one or more features, as well as to select one or more machine learning algorithms for selecting the features. Example features may include, without limitation, diagnostic data, treatment data, patient demographic data, patient geographic data, patient socioeconomic data, healthcare provider data, and/or healthcare provider reputation data. In some examples, the cohort may be defined based on one or more illnesses including, without limitation, breast cancer, non-small cell lung cancer, bone cancer, soft tissue sarcoma, colorectal cancer, central nervous system cancer, Hodgkin disease, and/or multiple myeloma.

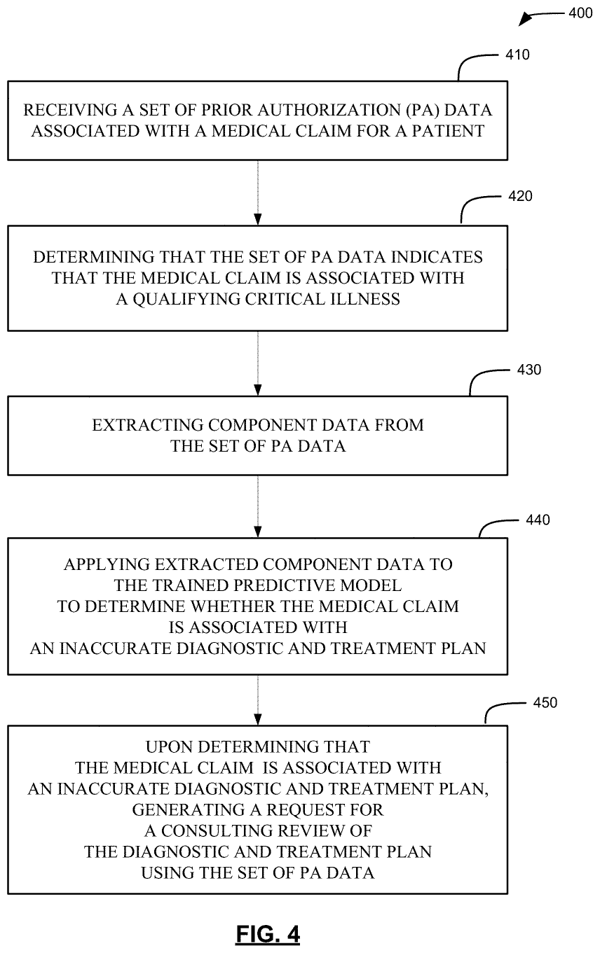

FIG. 4 is a flow diagram 400 representing a method for determining that a diagnosis and treatment plan is inaccurate from the perspective of the trained predictive server 310 (shown in FIG. 3). Specifically, trained predictive server 310 is configured to receive 410 a set of prior authorization (PA) data associated with a medical claim for a patient (e.g., claim data 124 or 324). For example, the PA data may include a diagnosis and treatment plan. In some examples, the trained predictive server 310 communicates with the first claim database server 120 to retrieve or collect other data associated with the medical claim and/or diagnosis and treatment plan (e.g., medical records, imaging, laboratory results, etc.).

Trained predictive server 310 may be configured to determine 420 whether the set of PA data indicates that the medical claim is associated with a qualifying critical illness (e.g., using coverage data 122 or 322). Trained predictive server 310 is further configured to extract 430 component data from the set of PA data. The features associated with the component data correspond to the features associated with the model 332. Example features may include, without limitation, diagnostic data, treatment data, patient demographic data, patient geographic data, patient socioeconomic data, healthcare provider data, and/or healthcare provider reputation data.

Trained predictive server 310 may be configured to apply 440 the extracted component data to the trained predictive model to determine whether the medical claim is associated with an inaccurate diagnosis and treatment plan. Based on the determination, the trained predictive server 310 may then determine an appropriate follow-up action. For example, if the medical claim is associated with an inaccurate diagnosis and treatment plan, the trained predictive server 310 may generate 450 a request for a consulting review of the diagnosis and treatment plan using the set of PA data. This enables the inaccurate diagnosis and treatment plan to be modified or updated with an accurate diagnosis and treatment plan. In some examples, the trained predictive server 310 communicates with the first claim database server 120 and/or first data warehouse server 320 to retrieve or collect other data associated with the medical claim and/or diagnosis and treatment plan (e.g., medical records, imaging, laboratory results, etc.) and transmits such data to a computing device associated with the consulting review provider along with the request for the consulting review or upon receiving an affirmative response to the request.

In some examples, the trained predictive server 310 maybe configured to identify or select a first consulting review provider from a list of candidate consulting review providers. The first consulting review provider may be selected based on candidate provider reputation data and candidate provider location data, for example. For example, the first consulting review provider may be geographically diverse (e.g., candidate provider location data is different from patient and/or healthcare provider).

To facilitate determining whether the medical claim is associated with an inaccurate diagnosis and treatment plan, the trained predictive server 310 may quantify the extracted component data by determining a likelihood of whether the medical claim is associated with an inaccurate diagnosis and treatment plan. In some examples, the trained predictive server 310 may determine that the medical claim is associated with an inaccurate diagnosis and treatment plan if the likelihood satisfies a predetermined threshold (e.g., the likelihood is greater than or equal to 80% or other suitable percentage). On the other hand, the trained predictive server 310 may determine that the medical claim is not associated with an inaccurate diagnosis and treatment plan (or is associated with an accurate diagnosis and treatment plan) when the likelihood does not satisfy a predetermined threshold (e.g., the likelihood is less than or equal to 20% or other suitable percentage). In some examples, the trained predictive server 310 may generate or transmit an alert to request additional input (e.g., from a systems analyst, physician, or other technical or health care professional) regarding whether the medical claim is associated with an inaccurate diagnosis and treatment plan when the likelihood is between a lower predetermined threshold (e.g., 20% or other suitable percentage) and an upper predetermined threshold (80% or other suitable percentage).

Example systems and methods for using trained predictive modeling to reduce misdiagnoses of critical illnesses are described herein and illustrated in the accompanying drawings. This written description uses examples to disclose aspects of the disclosure and also to enable a person skilled in the art to practice the aspects, including making or using the above-described systems and executing or performing the above-described methods. Examples described herein ensure predictive accuracy and facilitate providing improved workflows, runtime performance, and data quality. By identifying cases which are likely to benefit from a clinical consultation (or second opinion) review prior to treatment, the risk of improper, unwarranted, and/or missed treatment can be mitigated, and the quality and cost of healthcare can be significantly improved.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

When introducing elements of the disclosure or the examples thereof, the articles “a,” “an,” “the,” and “said” are intended to mean that there are one or more of the elements. References to an “embodiment” or an “example” of the present disclosure are not intended to be interpreted as excluding the existence of additional embodiments or examples that also incorporate the recited features. As used herein, the phrase at least one of A, B, and C should be construed to mean a logical (A OR B OR C), using a non-exclusive logical OR, and should not be construed to mean “at least one of A, at least one of B, and at least one of C.”

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements.

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A. The term subset does not necessarily require a proper subset. In other words, a first subset of a first set may be coextensive with (equal to) the first set.

In this application, including the definitions below, the term “module” or the term “controller” may be replaced with the term “circuit.” The term “module” may refer to, be part of, or include processor hardware (shared, dedicated, or group) that executes code and memory hardware (shared, dedicated, or group) that stores code executed by the processor hardware.

The module may include one or more interface circuits. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2016 (also known as the WIFI wireless networking standard) and IEEE Standard 802.3-2015 (also known as the ETHERNET wired networking standard). Examples of a WPAN are the BLUETOOTH wireless networking standard from the Bluetooth Special Interest Group and IEEE Standard 802.15.4.

The module may communicate with other modules using the interface circuit(s). Although the module may be depicted in the present disclosure as logically communicating directly with other modules, in various implementations the module may actually communicate via a communications system. The communications system includes physical and/or virtual networking equipment such as hubs, switches, routers, and gateways. In some implementations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various implementations, the functionality of the module may be distributed among multiple modules that are connected via the communications system. For example, multiple modules may implement the same functionality distributed by a load balancing system. In a further example, the functionality of the module may be split between a server (also known as remote, or cloud) module and a client (or, user) module.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave). The term computer-readable medium is therefore considered tangible and non-transitory. Non-limiting examples of a non-transitory computer-readable medium are nonvolatile memory devices (such as a flash memory device, an erasable programmable read-only memory device, or a mask read-only memory device), volatile memory devices (such as a static random access memory device or a dynamic random access memory device), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Swift, Haskell, Go, SQL, R, Lisp, Java®, Fortran, Perl, Pascal, Curl, OCaml, Javascript®, HTMLS (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

What is claimed is:

1. A trained predictive server for determining that a diagnosis and treatment plan is inaccurate, the trained predictive server comprising a processor and a memory, said processor configured to:
receive a set of prior authorization (PA) data associated with a medical claim for a patient;
determine that the set of PA data indicates that the medical claim is associated with a qualifying critical illness;
extract component data from the set of PA data;
apply the extracted component data to a trained predictive model associated with the qualifying critical illness to determine whether the medical claim is associated with an inaccurate diagnosis and treatment plan; and
upon determining that the medical claim is associated with the inaccurate diagnosis and treatment plan, generate a request for a consulting review of the diagnosis and treatment plan using the set of PA data.

2. The trained predictive server of claim 1, wherein the processor is further configured to:
identify a list of qualifying critical illnesses from a storage device in communication with the trained predictive server; and
apply the list to the set of PA data to determine that the set of PA data indicates that the medical claim is associated with the qualifying critical illness.

3. The trained predictive server of claim 1, wherein the processor is further configured to:
identify a list of features for extraction; and
extract the component data, based on the list of features, from the set of PA data.

4. The trained predictive server of claim 1, wherein the processor is further configured to:
identify a consulting review provider based on the set of PA data; and
transmit the request for the consulting review of the diagnosis and treatment plan to a computing device associated with the consulting review provider.

5. The trained predictive server of claim 1, wherein the processor is further configured to:
determine a likelihood of whether the medical claim is associated with the inaccurate diagnosis and treatment plan;
compare the likelihood of whether the medical claim is associated with the inaccurate diagnosis and treatment plan against a predetermined threshold to evaluate inaccuracies in the diagnosis and treatment plan; and
on condition that the likelihood of whether the medical claim is associated with the inaccurate diagnosis and treatment plan is greater than the predetermined threshold, determine that the medical claim is associated with the inaccurate diagnosis and treatment plan.

6. The trained predictive server of claim 1, wherein the processor is further configured to:
determine a likelihood of whether the medical claim is associated with the inaccurate diagnosis and treatment plan;
compare the likelihood of whether the medical claim is associated with the inaccurate diagnosis and treatment plan against a predetermined threshold to evaluate inaccuracies in the diagnosis and treatment plan; and
on condition that the likelihood of whether the medical claim is associated with the inaccurate diagnosis and treatment plan is less than the predetermined threshold, generate a request that the medical claim be processed.

7. The trained predictive server of claim 1, wherein the processor is further configured to:
determine a likelihood of whether the medical claim is associated with the inaccurate diagnosis and treatment plan;
compare the likelihood of whether the medical claim is associated with the inaccurate diagnosis and treatment plan against a plurality of predetermined thresholds to evaluate inaccuracies in the diagnosis and treatment plan, the plurality of predetermined thresholds including an upper predetermined threshold and a lower predetermined threshold; and
on condition that the likelihood of whether the medical claim is associated with the inaccurate diagnosis and treatment plan is between the upper predetermined threshold and the lower predetermined threshold, transmit an alert to a secondary device to request input regarding whether the consulting review is needed.

8. A method for determining that a diagnosis and treatment plan is inaccurate and performed by a trained predictive server including a processor and a memory, the method comprising:
receiving a set of prior authorization (PA) data associated with a medical claim for a patient;
determining that the set of PA data indicates that the medical claim is associated with a qualifying critical illness;
extracting component data from the set of PA data;
applying the extracted component data to a trained predictive model associated with the qualifying critical illness to determine whether the medical claim is associated with an inaccurate diagnosis and treatment plan; and
upon determining that the medical claim is associated with the inaccurate diagnosis and treatment plan, generating a request for a consulting review of the diagnosis and treatment plan using the set of PA data.

9. The method of claim 8, further comprising identifying a list of qualifying critical illnesses from a storage device in communication with the trained predictive server, wherein the list of qualifying critical illnesses is applied to determine whether the set of PA data indicates that the medical claim is associated with the qualifying critical illness.

10. The method of claim 8, further comprising identifying a list of features for extraction, wherein the list of features for extraction is used to extract the component data from the set of PA data.

11. The method of claim 8, further comprising:
identifying a consulting review provider based on the set of PA data; and
transmitting the request for the consulting review of the diagnosis and treatment plan to a computing device associated with the consulting review provider.

12. The method of claim 8, further comprising:
determining a likelihood of whether the medical claim is associated with the inaccurate diagnosis and treatment plan;
comparing the likelihood of whether the medical claim is associated with the inaccurate diagnosis and treatment plan against a predetermined threshold to evaluate inaccuracies in the diagnosis and treatment plan; and
on condition that the likelihood of whether the medical claim is associated with the inaccurate diagnosis and treatment plan is greater than the predetermined threshold, determining that the medical claim is associated with the inaccurate diagnosis and treatment plan.

13. The method of claim 8, further comprising:
determining a likelihood of whether the medical claim is associated with the inaccurate diagnosis and treatment plan;
comparing the likelihood of whether the medical claim is associated with the inaccurate diagnosis and treatment plan against a predetermined threshold to evaluate inaccuracies in the diagnosis and treatment plan; and
on condition that the likelihood of whether the medical claim is associated with the inaccurate diagnosis and treatment plan is less than the predetermined threshold, generating a request that the medical claim be processed.

14. The method of claim 8, further comprising:
determining a likelihood of whether the medical claim is associated with the inaccurate diagnosis and treatment plan;
comparing the likelihood of whether the medical claim is associated with the inaccurate diagnosis and treatment plan against a plurality of predetermined thresholds to evaluate inaccuracies in the diagnosis and treatment plan, the plurality of predetermined thresholds including an upper predetermined threshold and a lower predetermined threshold; and
on condition that the likelihood of whether the medical claim is associated with the inaccurate diagnosis and treatment plan is between the upper predetermined threshold and the lower predetermined threshold, transmitting an alert to a secondary device to request input regarding whether the consulting review is needed.

15. A trained predictive system for determining that a diagnosis and treatment plan is inaccurate, said trained predictive system comprising:
a first claim database server comprising a database processor and a database memory, the database memory includes a set of prior authorization (PA) data associated with a medical claim for a patient, the database processor configured to determine that the set of PA data indicates that the medical claim is associated with a qualifying critical illness; and
a trained predictive server in communication with the first claim database server, the trained predictive server comprising a processor and a memory, the processor configured to:
receive the set of PA data associated with the medical claim for the patient from the first claim database server;
extract component data from the set of PA data; and
apply the extracted component data to a trained predictive model associated with the qualifying critical illness to determine whether the medical claim is associated with an inaccurate diagnosis and treatment plan; and
wherein the database processor is further configured to:
receive an indication that the medical claim is associated with the inaccurate diagnosis and treatment plan from the trained predictive server; and
generate a request for a consulting review of the diagnosis and treatment plan using the set of PA data.

16. The trained predictive system of claim 15 further comprising a first data warehouse server in communication with the first claim database server, the first data warehouse server comprising a data warehouse processor and a data warehouse memory;
wherein the database processor is further configured to:
receive a list of qualifying critical illnesses from the first data warehouse server; and
apply the list to the set of PA data to determine that the set of PA data indicates that the medical claim is associated with the qualifying critical illness.

17. The trained predictive system of claim 15, wherein the processor is further configured to:
identify a list of features for extraction; and
extract the component data, based on the list of features, from the set of PA data.

18. The trained predictive system of claim 15, wherein the database processor is further configured to:
identify a consulting review provider based on the set of PA data; and
transmit the request for the consulting review of the diagnosis and treatment plan to a computing device associated with the consulting review provider.

19. The trained predictive system of claim 15, wherein the processor is further configured to:
determine a likelihood of whether the medical claim is associated with the inaccurate diagnosis and treatment plan;
compare the likelihood of whether the medical claim is associated with the inaccurate diagnosis and treatment plan against a predetermined threshold to evaluate inaccuracies in the diagnosis and treatment plan; and
on condition that the likelihood of whether the medical claim is associated with the inaccurate diagnosis and treatment plan is greater than the predetermined threshold, determine that the medical claim is associated with the inaccurate diagnosis and treatment plan.

20. The trained predictive system of claim 15, wherein the processor is further configured to:
determine a likelihood of whether the medical claim is associated with the inaccurate diagnosis and treatment plan;
compare the likelihood of whether the medical claim is associated with the inaccurate diagnosis and treatment plan against a predetermined threshold to evaluate inaccuracies in the diagnosis and treatment plan; and
on condition that the likelihood of whether the medical claim is associated with the inaccurate diagnosis and treatment plan is less than the predetermined threshold, generate a request that the medical claim be processed.
